(19) United States
(12) Patent Application Publication
(10) Pub. No.: US 2020/0317518 A1
(43) Pub. Date: Oct. 8, 2020

(54) OZONE GENERATOR VOLTAGE VERIFICATION LIGHT ASSEMBLY

(71) Applicant: Michael P. Schmidt, Clarence, NY (US)

(72) Inventor: Michael P. Schmidt, Clarence, NY (US)

(21) Appl. No.: 16/375,167

(22) Filed: Apr. 4, 2019

(51) Int. Cl. C01B 13/11; A61L 9/04; A61L 9/12

(52) U.S. Cl. CPC C01B 13/115; A61L 9/046; A61L 9/12; A61L 2209/212; C01B 2201/34; C01B 2201/24; A61L 2209/11; C01B 2201/12

(57) ABSTRACT

An ozone generator voltage verification light assembly generates a visual or audible indicator, such as a neon light, to verify that an ozone generator is generating the proper voltage to generate ozone, and/or ozone generator voltage verification light assembly testing assembly operatively connects to an ozone generator, and visually indicates if an irregularity in voltage occurs. The ozone generator includes a power source for supplying electrical current, and a transformer that generates high voltage. The ozone generator also includes single or multiple ceramic plates disposed in a spaced-apart, parallel relationship, and coated with stainless steel mesh. Voltage generated by the transformer contacts the ceramic plates by means of an electrode. Accordingly, ozone is generated by discharging electricity through electrodes that contact in both sides of ceramic plate.

### FIELD OF THE INVENTION

The present invention relates generally to an ozone generator voltage verification light assembly. More so, the present invention relates to a testing assembly that provides a visual alert mechanism and/or an audible alert mechanism, such a neon light that operatively connects to an ozone generator for verification that the generator is producing the proper voltage to generate ozone; thereby allowing a user to quickly and easily determine what is wrong with the ozone generator when it no longer is producing ozone.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, an ozone generator is a device that turns ambient air into ozone, which is capable of removing contaminants in the air. Ozone generators intentionally produce ozone and are sold as air cleaners for commercial and residential applications. Specifically, they are advertised to deodorize, disinfect, kill or remove dangerous or irritating airborne particles in indoor environments.

Generally, ozone is a naturally occurring gas composed of three oxygen molecules and not two. The third molecule can detach and apply itself to other chemicals or substances. This ability to attach to other substances means ozone is able to alter these substances. This is why ozone is generated and used for certain purposes. Further, it is known that ozone has a half-life of about 30 minutes in at ambient temperatures.

Other proposals have involved ozone generators. The problem with these ozone generators is that they do not have a way to easily diagnose when the electrical circuitry has a malfunction or when the ceramic plates are depleted, broken, or need to be cleaned. Even though the above cited ozone generators meet some of the needs of the market, an ozone generator voltage verification light assembly that provides a visual alert mechanism and/or an audible alert mechanism, such as an neon light, that operatively connects to an ozone generator for verification that the ozone generator is generating the proper voltage to generate ozone, and that the ceramic plate is depleted or dirty, so that the user can quickly determine what is wrong with the ozone generator when it no longer is producing ozone. Furthermore, the current proposal is not obvious because the high voltage makes it impossible to use any normal types of lights or warning systems. Those skilled in the art will recognize that a neon light with a 120K limiting resistor is the only possible way to provide a visual aid to determine if the ozone generator is operating properly.

### SUMMARY

Illustrative embodiments of the disclosure are generally directed to an ozone generator voltage verification light assembly. The assembly provides a visual alert mechanism and/or an audible alert mechanism, such as a neon light, that operatively connects to an ozone generator for verification that the ozone generator is generating the proper voltage to generate ozone. The visual or audible alert mechanism verifies that the ozone generator is operating correctly. In this manner, the ozone generator can continue deodorizing and sterilizing the ambient air.

The ozone generator voltage verification light assembly is configured to operatively connect to an ozone generator, and visually and/or audibly indicate if an irregularity in voltage occurs, so that repairs and replacements can be made. The ozone generator includes a power source for supplying an electrical current, a transformer that generates high voltage by boosting voltage supplied from power. The ozone generator may also include one or more ceramic plates, (UV bulbs, or other dielectrics) disposed in a spaced-apart, parallel relationship, and coated with an electrode. The ozone plate also includes stainless steel mesh on both sides of the ceramic plate.

A predetermined voltage generated by the high voltage transformer is applied to the stainless steel mesh attached to the outside of the ceramic plate(s). Accordingly, the ozone generator generates ozone by discharging electricity to both sides of the ceramic plate(s). The ozone generator voltage verification light assembly monitors the predetermined voltage. If the predetermined voltage exceeds a range (too low or too high), the neon light turns off, thereby alerting the user that the unit has malfunctioned.

In another aspect, the ozone generator comprises a housing.

In another aspect, the ozone generator comprises a handle joined to the housing.

In another aspect, the transformer generates more than 3 volts.

In another aspect, the power source comprises an AC power supply.

In another aspect, the ozone generator comprises a stack-plate design, but may be made of other dielectrics.

In another aspect, the electrical current comprises AC current.

In another aspect, the ozone generator may have multiple slots sized and dimensioned to receive the ceramic plates.

In another aspect, the ceramic ozone plate(s) is coated with stainless steel mesh.

In another aspect, the voltage testing (verification) device is a 220 volt neon light with a resistance of 120K.

One objective of the present invention is to generate ozone for deodorizing, disinfecting, killing, and removing dangerous or irritating airborne particles in indoor environments.

Another objective is to have a visual and/or audible verification that the ozone generator is operating properly.

Another objective is to verify a constant source of voltage to the ceramic plates.

Yet another objective is to ensure that the ceramic plates are replaced or cleaned when depleted or dirty.

An exemplary objective is to provide an inexpensive and easy verification that the ozone generator is working properly.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a rear perspective view of an exemplary ozone generator voltage verification light assembly, in accordance with an embodiment of the present invention;

FIG. 2 illustrates a front perspective view of an exemplary ozone generator voltage verification light assembly, in accordance with an embodiment of the present invention;

FIG. 3 illustrates a perspective view of the inner electrical components in the housing of the ozone generator, in accordance with an embodiment of the present invention;

FIG. 4 illustrates a close up view of the visual alert mechanism, or neon light, in accordance with an embodiment of the present invention;

FIG. 5 illustrates a perspective view of a ceramic plate being pulled from the ozone generator, with the visual alert mechanism (verification system) deactivated, in accordance with an embodiment of the present invention;

FIG. 6 illustrates a perspective view of the ceramic plate from FIG. 5 sliding into the ozone generator, with the visual alert mechanism (verification system) deactivated, in accordance with an embodiment of the present invention; and

FIG. 7 illustrates a perspective view of the ceramic plate from FIG. 5 fully inserted into the body of the ozone generator, in accordance with an embodiment of the present invention.

Like reference numerals refer to like parts throughout the various views of the drawings.

### DETAILED DESCRIPTION OF THE INVENTION

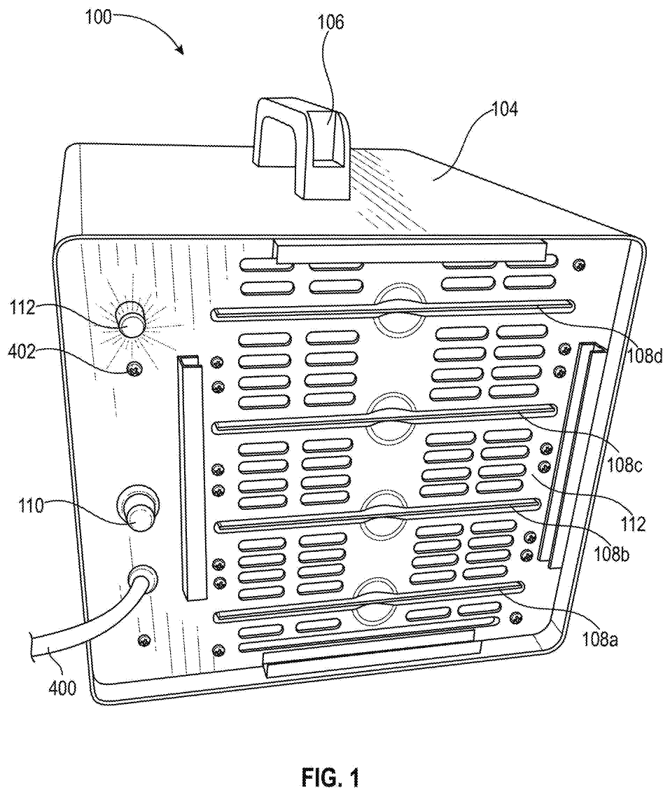

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

An ozone generator 100 is referenced in FIGS. 1-7. The ozone generator is configured to emit a visual and/or audible alert when an ozone generator 102 is operating properly. The alert provides an immediate indication that the ozone generator 100 is operating properly. Specifically, since the ozone generator 100 requires voltage to pass to ceramic plates 108a-d in order to generate ozone, the alert is an indication that the voltage is adequate for production of ozone. This allows a user to quickly and easily determine the cause of a malfunction without the need of any type of diagnosis. If the ozone generator voltage light assembly 112 is illuminated and the ozone generator 100 is not producing ozone, the only possible reason is that the ozone plate(s) 108a-db is/are depleted or dirty.

As referenced in FIG. 1, the ozone generator 100 turns ambient air into ozone. As is known in the art, ozone is used to remove contaminants in the ambient air, creating an effect on some sources of odor inside a closed space. Specifically, the ozone generator 100 intentionally produces ozone, which serves as an air cleaner for commercial and residential applications. In this manner, the ozone generator 100 can deodorize, disinfect, kill or remove dangerous or irritating airborne particles in indoor environments.

Those skilled in the art will recognize that ozone is oxygen but in a different form. Ozone contains three oxygen molecules and not two. The third molecule can detach and apply itself to other chemicals or substances, thereby creating a safer, cleaner, odor free environment.

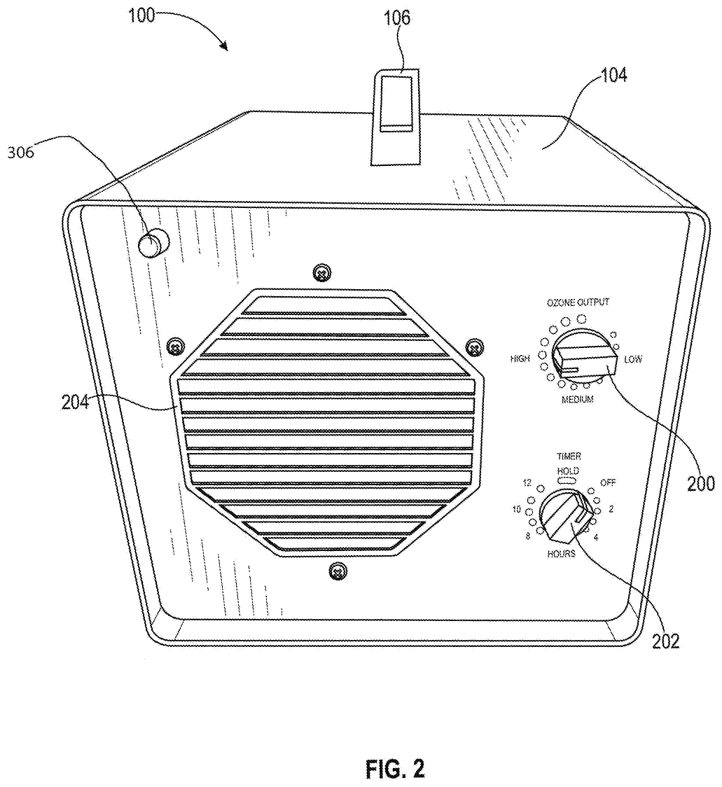

The ozone generator 100 comprises a housing 104 with a handle 106 that allows for carrying the housing 104. The housing 104 can be a metal or rigid polymer housing that protects the internal components from damage, moisture, and debris. The housing 104 can have a rectangular shape as shown in FIG. 2, or may have other shapes and sizes the present invention is scalable such that the size and shape can have myriad combinations.

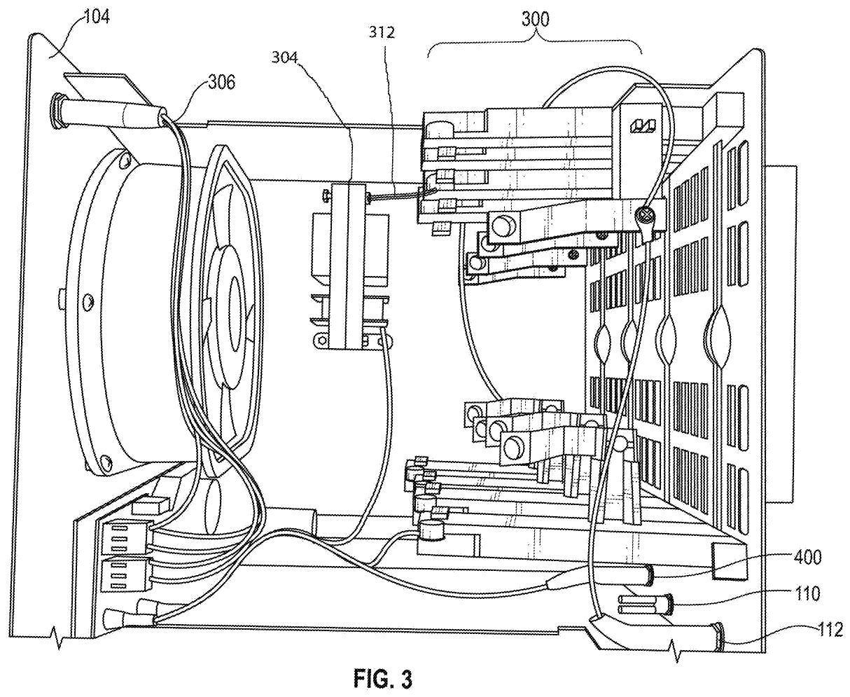

Turning now to FIG. 3, the ozone generator 100 comprises a power source 400 that supplies an electrical current and passes through the fuse 110. In one non-limiting embodiment, the power source 400 comprises 110-120 Volt AC power supply. A fuse 110 may be operatively connected to the power source 400 as an electrical safety device that operates to provide overcurrent protection of an electrical circuit. When the ozone generator is powered on, the power indicator light 306 is illuminated and verifies that the unit is operating.

The ozone generator 100 may also provide a transformer 304 generating high voltage by boosting voltage supplied from the power source 400. The high voltage transformer 304 generates more than 3 volts. A timer switch 202 is operable to regulate the duration by which ozone is generated. A variable ozone output switch 200 regulates the voltage passing through the ceramic plates 108a-d, as described below. An ozone outlet 204 provides a large opening for the generated ozone to be released from the ozone generator 100.

As shown in FIG. 1, a chief component of the ozone generator 100 is the ceramic plate(s) 108a-d that are disposed in a spaced-apart, parallel relationship. The ceramic plates may be disposed in a stacked, parallel arrangement. In one embodiment, the ozone generator 100 comprises a stack-plate design that carries the ceramic plates in this manner.

In some embodiments, the ceramic plates 108a-d may be coated with an electrode. The electrode that coats the ceramic plates 108a-d may include, without limitation, alumina and a sintered metal electrode. Accordingly, the ozone generator 100 generates ozone by discharging electricity through the electrodes which contact both sides of the ceramic plates 108a-d.

In some embodiments, the ozone generator 100 provides a plastic ceramic plate receiver 300 that encapsulates the ceramic plates 108a-d, retaining the plates in the stacked arrangement. The plastic ceramic plate receiver 300 may comprise single or multiple slots sized and dimensioned to receive the ceramic plates 108a-d. Voltage generated by the high voltage transformer 304 passes to the ozone plates 108a-d via that are secured by the plastic ceramic plate receiver 300. Accordingly, the ozone generator 100 generates ozone by discharging electricity through electrodes which contact both sides of ceramic plates 108a-d. Suitable materials for the plastic ceramic plate receiver 300 may include, without limitation, plastic, stainless steel, titanium, and metal alloys.

In operation, when the ozone generator 100 is operational to produce ozone, a predetermined voltage is generated by the transformer 304. The predetermined voltage passes to the ceramic plates 108a-d, via a high voltage wire 312. In one non-limiting embodiment of the predetermined voltage is between 1600 and 4800 volts. Though more or less volts may be used depending on the amount of ozone required, the number of ceramic plates 108a-d, and the size of the ozone generator 100.

In some embodiments, the ozone generator 100 provides an electrode operatively connected to the plastic ceramic plate receiver 300 and the ceramic plates 108a-d. The electrode.

For the present invention, the ozone generator voltage verification light assembly 112 determines if the generated voltage is within a range of the predetermined voltage. The range may include 0.2 volts above or below the predetermined voltage. However, the range may be more or less than 0.2 volts, depending on the type of ozone generator 100 and the specifications of the ceramic plates 108a-d. In any case, the verification alert signal is transmitted when the range of the predetermined voltage for the specific ozone generator 102 is exceeded.

In one possible embodiment, the ozone generator voltage verification light assembly 112 includes a ground wire, a 220 volt neon light, a power source and a power wire. The ozone generator voltage verification light assembly 112 is operatively connected to the ceramic plate, so as to detect anomalies therein. Thus, if the detected voltage exceeds a range of the predetermined voltage, the ozone generator voltage verification light assembly 112 generates an alert signal, by turning off. The proper voltage must pass through the electrode, or the ozone generator voltage verification light assembly 112 alerts accordingly. When the transformer 304 or other electrical components of the ozone generator 102 are not operating properly and thereby generating or carrying the appropriate voltage or current, the range of the predetermined voltage is exceeded, which results in the ozone generator voltage verification light assembly 112 alert signal being generated. Also, the viability of the ceramic plate 108a-d, is determined by the ozone generator voltage verification light assembly 112.

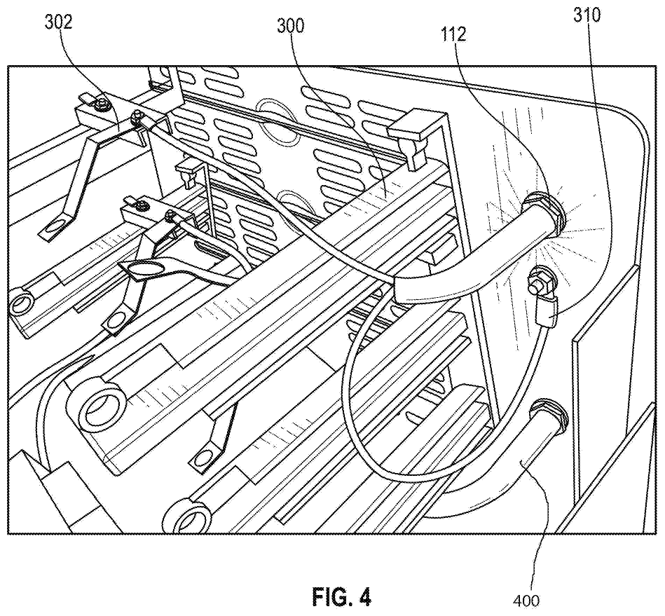

Looking now at FIG. 4, the ozone generator 100 provides an ozone generator voltage verification light assembly 112 that is operatively connected to the electrode 302. The ozone generator voltage verification light assembly 112 is deactivated when a predetermined voltage is exceeded. The ozone generator voltage verification light assembly 112 may include a neon light. Thus, the neon light will remain illuminated as long as the ozone generator 100 is operating properly. The neon light will light up constantly and will remain illuminated as long as the ozone generator 100 is operating properly. In any case, the proper voltage must pass through the electrode, or the ozone generator voltage verification light assembly 112 alerts accordingly, by turning off. A ground wire 303 is also connected to a grounding screw 402.

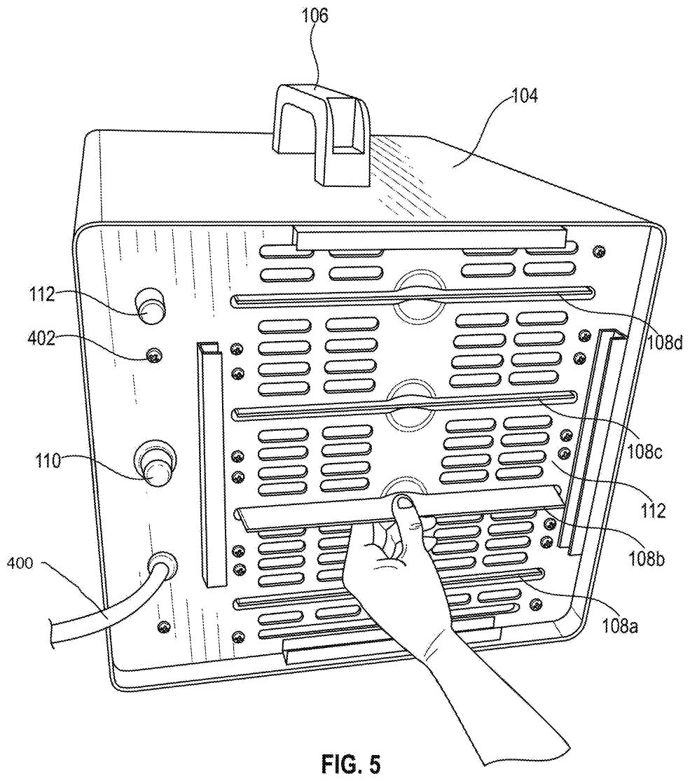

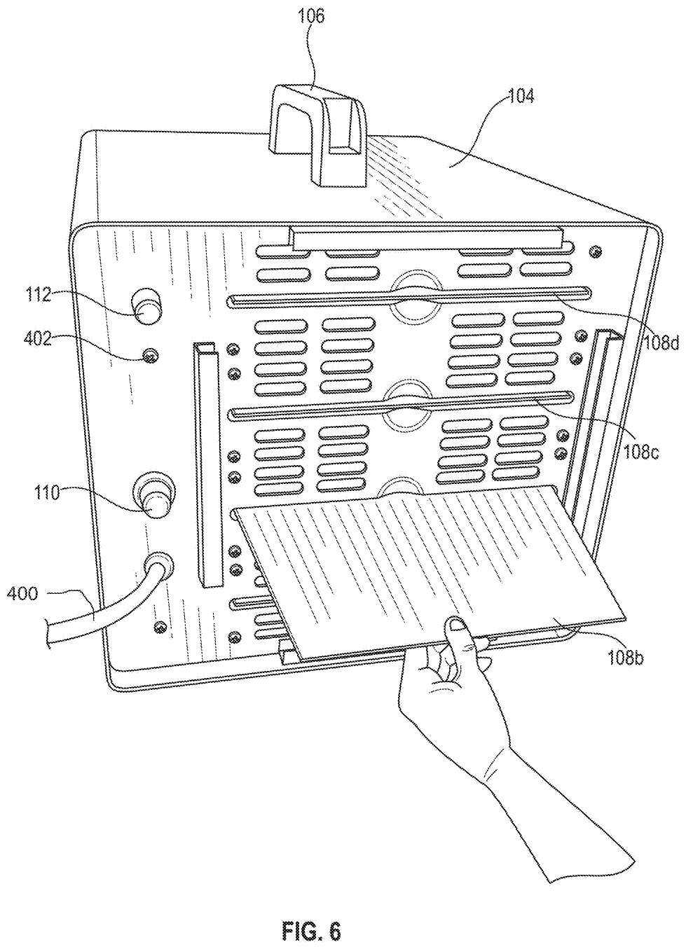

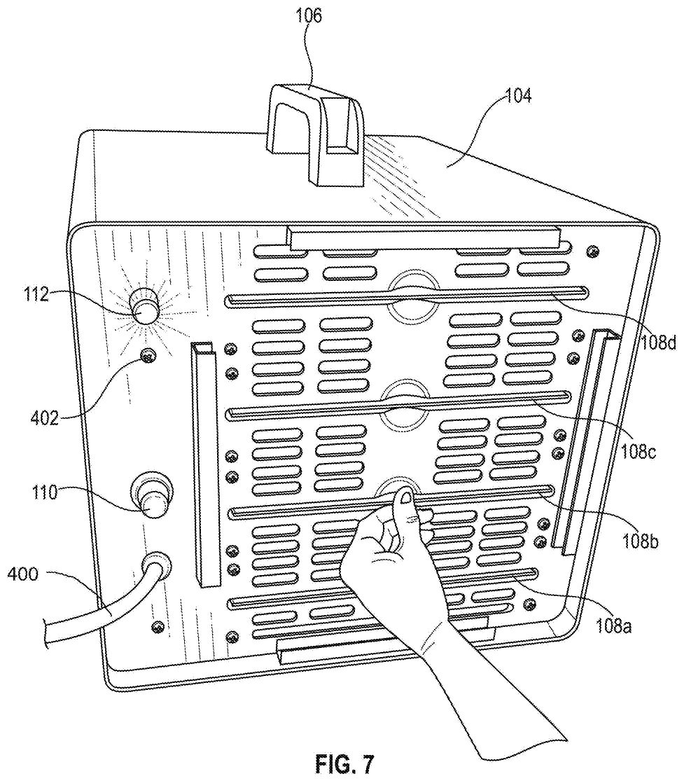

FIG. 5 illustrates a perspective view of a ceramic plate being pulled from the ozone generator 100, with the ozone generator voltage verification light assembly 112 deactivated. FIG. 6 illustrates a perspective view of the ceramic plate from FIG. 5 sliding into the ozone generator 100, with the ozone generator voltage verification light assembly 112 deactivated. FIG. 7 illustrates a perspective view of the ceramic plate fully inserted into the plastic ceramic plate receiver 300 and operational in the ozone generator 102.

In an alternative embodiment, the ozone generator 100 may include an audible alert mechanism that is operatively connected to the ozone generator voltage verification light assembly 112. Similar to the visual alert mechanism, the audible alert mechanism is actuated when the alert signal is detected. The optional audible alert mechanism would help verify that the ozone generator 100 and ceramic plates 108a-d are operating correctly.

In this manner, the ozone generator 100 can continue deodorizing and sterilizing the ambient air. In essence, the ozone generator voltage verification light assembly 112 measures a predetermined voltage range in an ozone generator 100, and then illuminates a neon light to indicate the operability or range of voltage across the ceramic plates 108a-d in the ozone generator 100.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. An ozone generator voltage verification light assembly, the assembly comprising:
an ozone generator operable to produce ozone, the ozone generator having:
a power source supplying an electrical current;
a transformer generating high voltage by boosting voltage supplied from the power source;
at least one ceramic plate disposed in a spaced-apart, parallel relationship;
a metal housing encapsulating the ceramic plate; and
whereby, when the ozone generator is operational to generate ozone, a predetermined voltage is generated by the transformer, the predetermined voltage contacting both sides of the ozone plate via an electrode.

2. The assembly of claim 1, wherein the assembly operatively connects to the metal case and the electrode that supplies power to the ceramic plate, the assembly detects the voltage passing to the ceramic plate, the assembly generates an alert signal by turning off if the detected voltage exceeds a range of the predetermined voltage.

3. The assembly of claim 1, further comprising a metal housing and a handle.

4. The assembly of claim 3, wherein the metal housing forms multiple slots sized and dimensioned to receive the ceramic plate.

5. The assembly of claim 1, wherein the ozone generator comprises a stack-plate design.

6. The assembly of claim 1, wherein the transformer generates more than 3 volts.

7. The assembly of claim 1, wherein the power source comprises AC power.

8. The assembly of claim 1, wherein the ozone generator produces more than 0.05 g/hour of ozone.

9. The assembly of claim 1, wherein the ozone generator assembly is made of ceramic ozone plates with stainless steel mesh attached to both sides, or comprises a UV bulb, an ozone card, plasma, Rods, or another dielectric that causes ozone to be produced in ambient air.

10. The assembly of claim 1, wherein an electrode coating the ceramic plate comprises stainless steel.

11. The assembly of claim 1, wherein the predetermined voltage comprises more than 3 volts.

12. The assembly of claim 1, further comprising a neon light.

13. The assembly of claim 1, further comprising an audible alert mechanism operatively connected to the ozone generator voltage verification light assembly, the audible alert mechanism being actuated when an alert signal is detected.

14. An ozone generator voltage verification light assembly, the assembly comprising:
an ozone generator operable to produce ozone, the ozone generator having:
a housing;
a handle joined to the housing;
a power source supplying an electrical current;
a high voltage transformer generating high voltage by boosting voltage supplied from the power source;
at least one ceramic plate disposed in a spaced-apart, parallel relationship, the ceramic plates being coated with an electrode;
stainless steel mesh encapsulating the ceramic plate;
whereby, when the ozone generator is operational to generate ozone, a predetermined voltage is generated by the transformer, the predetermined voltage passing through the electrode and to the ceramic plate;
an ozone generator voltage verification light assembly operatively connected to the housing and the electrode that supplies the ceramic plates with high voltage power, the ozone generator voltage verification light assembly detecting the voltage passing through the electrode and to the ceramic plates, the ozone generator voltage verification light assembly turning off if the detected voltage exceeds a range of the predetermined voltage; and
a visual alert mechanism being actuated when an alert signal is detected.

15. The assembly of claim 14, wherein the visual alert mechanism comprises a neon light.

16. An ozone generator voltage verification light assembly, the assembly comprising:
an ozone generator operable to produce ozone, the ozone generator having:
a housing;
a power source supplying an electrical current;
a high voltage generator generating high voltage by boosting voltage supplied from the power source;
multiple ceramic plates disposed in a spaced-apart, parallel relationship, the ceramic plates being coated with an electrode;
a stainless steel mesh encapsulating the ceramic plate;
whereby, when the ozone generator is operational to generate ozone, a predetermined voltage is generated by the high voltage generator, the predetermined voltage passing to the stainless steel mesh attached to the ceramic plates;
a voltage detector operatively connected to the metal cage and the ceramic plates, the voltage detector detecting the voltage passing through the metal cage and between the ceramic plates, the voltage detector generating an alert signal if the detected voltage exceeds a range of the predetermined voltage; and
a visual alert mechanism operatively connected to the voltage detector, the visual alert mechanism being actuated when the alert signal is detected.

17. The assembly of claim 16, wherein the visual alert mechanism comprises a neon light.

18. The assembly of claim 16, further comprising an audible alert mechanism operatively connected to the voltage detector, the audible alert mechanism being actuated when the alert signal is detected.

19. The assembly of claim 16, further comprising an oscillator generating an oscillation frequency.

20. The assembly of claim 16, further comprising a transmitter that relays the alert signal from the voltage detector to a remote communication device.
